(19) United States
(12) Patent Application Publication
(10) Pub. No.: US 2023/0313306 A1
(43) Pub. Date: Oct. 5, 2023

(54) Methods of Diagnosis of Alzheimer's Disease by Monitoring STING Signaling Activation

(71) Applicant: Feng Xue, McKinney, TX (US)

(72) Inventor: Feng Xue, McKinney, TX (US)

(21) Appl. No.: 18/186,897

(22) Filed: Mar. 20, 2023

Related U.S. Application Data
(60) Provisional application No. 63/321,779, filed on Mar. 21, 2022.

(51) Int. Cl. C12Q 1/6883; C12Q 1/686; C12Q 1/6827

(52) U.S. Cl. CPC C12Q 1/6883; C12Q 1/6827; C12Q 1/686; C12Q 2600/156

(57) ABSTRACT

The disclosed embodiments relate the diagnosis of Alzheimer’s Disease (AD) by monitoring STING (TMEM173) signaling activation in brain immune system and peripheral immune system. Our study shows that, STING signaling activation is one of the major features of early neuroinflammation and microglia (Mg) pro-inflammatory activation in mild AD or at early stage of AD. In late neuroinflammation of moderate and severe AD, STING signaling activation can also be detected in the Mg subgroup, the Interferon-Related Modules. And the STING signaling activation is induced by A β oligomer and can also be detected in leukocytes and white blood cells. Therefore, the STING signaling activation is a diagnosis marker of mild, moderate, and severe AD.

### RELATED APPLICATIONS

The present application claims priority to and is a non-provisional of U.S. Provisional Application Ser. No. 63/321,779 filed Mar. 21, 2022, all of which is incorporated herein by reference.

### TECHNICAL FIELD

The invention relates the technical field of diagnosis research of Alzheimer’s Disease (AD), in particular the application of monitoring STING (TMEM173) signaling activation in diagnosis of AD.

### BACKGROUND

Alzheimer’s Disease (AD) is a degenerative disease of the central nervous system characterized by progressive cognitive dysfunction and behavioral impairment. According to the 2015 World Alzheimer’s Report, there are about 46.8 million people with AD worldwide, with an average of one new case occurring every three seconds. It is predicted that the number of people with AD will exceed 131.5 million in 2050.

AD is a central neurodegenerative disease with high mortality. In recent years, its incidence rate has been increasing rapidly, which has caused serious social burden. Chronic neuroinflammation is an important pathological feature of AD, therefore, is a good target for AD diagnosis.

Recently, early neuroinflammation in mild AD were revealed in clinical and animal model studies [1]. Our study shows that, in mild AD, before the plaque accumulation of Aβ amyloid protein [2], blocking (e.g., gene knockout) or decreasing ( e.g., small molecule antagonist treatment) STING signaling activation can significantly reduce the pro-inflammatory activation of microglia (Mg) in the young AD mouse models (2-3 months old). It can also reduce proliferation of the pro-inflammatory Mg cells, decrease late neuroinflammation in the old AD mouse models, and partially improve the cognitive ability of AD mouse models. Therefore, STING signaling activation is an important feature of early neuroinflammation and Mg pro-inflammatory activation in mild AD or at early stage of AD.

In late neuroinflammation of moderate and severe AD, STING signaling activation can also be detected in the Mg subgroup, the Interferon-Related Modules [7]. This subgroup locates far away from the Aβ plaque and can be detected by single cell RNAseq (scRNAseq) or other single cell technologies. Therefore, the STING signaling activation is also an important feature in AD late neuroinflammation and can be used as a diagnosis marker in a cellular subgroup to identify AD.

STING signaling pathway was firstly defined as an early immune signaling pathway that activates antiviral Interferon I (IFN I) in cells in the early stage of viral infection [3]. Recent studies have shown that IFN I is one of the upstream pro-inflammatory immune signaling pathways in AD chronic neuroinflammation, and can directly induce Mg to engulf synapses of neurons [4,5]. Our new study revealed that STING signaling pathway is one of the major upstream synthesis pathways of IFN I in AD chronic neuroinflammation, and is one of the major Mg pro-inflammatory immune signaling pathway in mild AD before the accumulation of Aβ amyloid plaques. The in vitro cellular assay revealed that the STING signaling activation is induced by Aβ oligomer and can also be detected in leukocytes and white blood cells. Therefore, STING signaling activation is a diagnosis marker of mild, moderate, and severe AD.

### SUMMARY

One embodiment discloses a diagnosis method of Alzheimer’s Disease (AD) by monitoring STING (TMEM173) signaling activation in brain immune system and peripheral immune system.

One embodiment relates the preparation of products for diagnosis of AD by monitoring STING signaling activation. The AD can be mild, moderate, or severe.

STING signaling pathway refers to either the cGAS-STING-TBK1-IRF3-IFN I cascade signaling pathway or the cGAS-STING-IKK-NFkB-IFN I cascade signaling pathway or both.

The methods for monitoring STING signaling activation refers to qRT-PCR, Digital qPCR, Next generation sequencing (NGS), Single cell RNAseq (scRNAseq), In situ hybridization, North blot, Electrophoretic mobility shift assay (EMSA), Proteomics, Western blot, Antibodies staining, Immunohistochemistry, Immunocytochemistry, Immunofluorescence (IF), Bioinformatics, Immunoprecipitation (IP), Co-immunoprecipitation (Co-IP), Enzyme-linked immunosorbent assay (ELISA), Fluorescence resonance energy transfer (FRET), Reporting gene assay, siRNA, miRNA, antisense oligonucleotides, CRISPR/Cas9 gene editing products, antagonists, blockers, small molecule compounds that inhibit the function and/or activity of main molecules of STING signaling pathways, and one of or a combination of promoter elements or expression vectors, which can downregulate the gene, the mRNA, or the protein expression levels of the major molecules of the STING signaling pathway..

Monitoring STING signaling activation targets human STING signaling pathway.

The products include reagents, enzymes, kits, devices, equipment, and their compositions.

The detection ways of monitoring STING signaling activation includes both in vitro detection and in vivo detection.

### BRIEF DESCRIPTION OF THE DRAWINGS

FIG. 1 are schematic diagrams of the changes of the downstream signaling pathways by blocking or decreasing STING signaling activation.

The FIGURE is a collection of charts providing experimental results data in one embodiment.

The FIGURE is a collection of charts providing experimental results data in one embodiment.

The FIGURE is a collection of charts providing experimental results data in one embodiment.

The FIGURE is a collection of charts providing experimental results data in one embodiment.

The FIGURE is a collection of charts providing experimental results data in one embodiment.

The FIGURE is a collection of charts providing experimental results data in one embodiment.

### DETAILED DESCIPTION

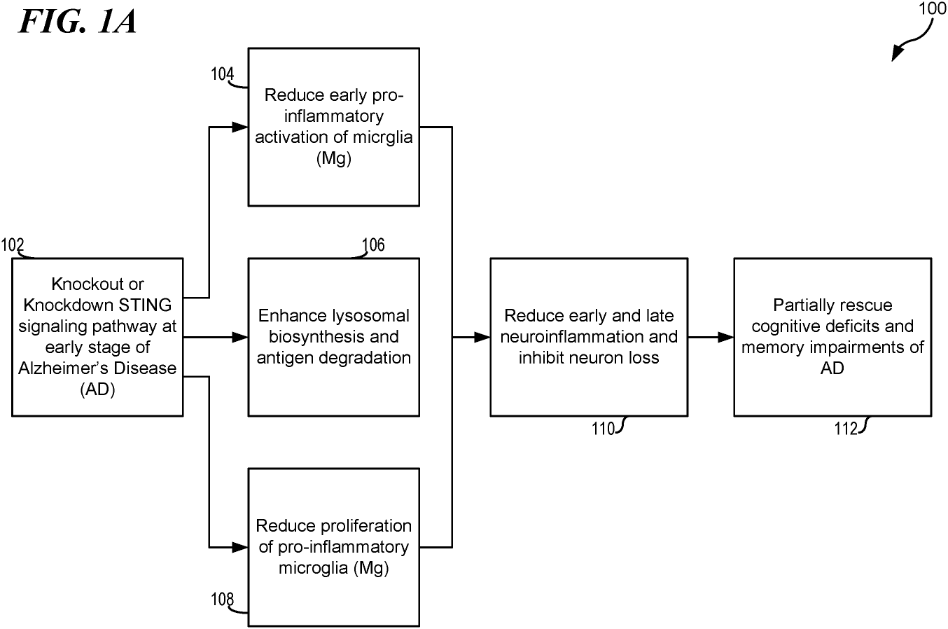

Referring to FIG. 1, presented is a schematic diagram of the changes of the downstream signaling pathways by blocking or decreasing STING signaling activation is illustrated.

As shown in block 102, one embodiment includes first comprises knockout or knockdown of a STING signaling pathway at an early stage of Alzheimer’s Disease (AD).

As shown in block 104, the illustrated embodiment includes reducing early pro-inflammatory activation of microglia (Mg).

As shown in block 106, the illustrated embodiment includes enhancing lysosomal biosynthesis and antigen degradation of microglia (Mg).

As shown in block 108, the illustrated embodiment includes reducing proliferation of pro-inflammatory microglia (Mg).

In one embodiment, the actions depicted in blocks 104, 106, and 108 are performed in any order with substantially overlapping execution times. In a preferred embodiment, the actions depicted in blocks 104, 106, and 108 are performed simultaneously. In one embodiment, actions performed simultaneously do not require strictly contemporaneous start and end times. In one embodiment, actions performed simultaneously can vary in execution times and such actions are performed simultaneously if the execution start and end times at least partially overlap for a majority of the actions.

As shown in block 110, the illustrated embodiment next reduces early and late neuroinflammation and inhibits neuron loss. Generally, the action depicted in block 110 occurs after the completion of active efforts in the actions depicted in blocks 104, 106, and 108.

As shown in block 112, the illustrated embodiment next partially rescues cognitive deficits and memory impairments of AD.

Experimental approaches confirmed the validity of the embodiments disclosed herein and in particular the embodiments as recited in the claims below.

Human brain tissue were purchased from NIH NeuroBioBank. Human peripheral blood monocytes (HBMs) were isolated from the blood of 6 healthy donors or the blood of 6 AD patients using standard Ficoll-Hypaque gradient centrifugation. The monocytes were counted in a hemocytometer and were seeded in a 24-well plate at concentration of 10,000 cells per cm². The cells were cultured in RPMI 1640 medium (supplemented with 10% heat-inactivated fetal bovine serum, 100 mg/ml penicillin, and 100 mg/ ml streptomycin) and incubated overnight at 37° C. under a humidified atmosphere containing 5% CO2. After incubation, the tested monocytes were treated with STING antagonist C-178 [6] with 1 µM final concentration for 6 hr and then were collected for transcriptomics or qRT-PCR analysis. In qRT-PCR analysis, for a cell sample of each donor, 3 biological replicates (wells) were designed and the significance was evaluated by Student’s t-Test in Microsoft EXCEL software. The average value of the 3 biological replicates was defined as the relative expression level of the cell sample of the donor.

As the immune cytokine mRNAs without polyA or with short polyA can not be captured in the typical polyA-selected RNA-Seq library method, the whole transcriptomics RNAseq library were prepared with rRNA depletion method. Briefly, the brain tissue or peripheral blood mononuclear cells were fully lysed in TRIzol (Invitrogen) and total RNA were purified by ZYMO Direct-zol RNA Microprep Kits (ZYMO). Purified total RNA were directly used for rRNA depletion and RNAseq library preparation using TaKaRa SMARTer Stranded Total RNA-Seq Kit v2 - Pico Input Mammalian. Total RNA and RNAseq library were quantified by Qubit 3.0 Fluorometer. The RNA integrity and cDNA fragments were analyzed by AATI Fragment Analyzer. The libraries were multiplexed and then sequenced on Illumina Nextseq 500 (Illumina) to generate 33M of single end 75 base pair reads per library.

Quantitative RT-PCR (qRT-PCR) was used to determine gene expression of mRNA markers in AD mouse models or human samples. The tissue or cell samples were fully dissolved in TRizol (Invitrogen, 15596018). The tissue samples were thoroughly ground in 1.5 mL tubes with RNase/Dnase-free pestle. Total RNA was purified by ZYMO Direct-Zol RNA Microprep Kits (ZYMO, R2063) and cDNA was synthesized by PrimeScript RT Master Mix (TaKaRa, RR036A). The cDNA was then preamplified with PerfeCTa PreAmp SuperMix (Quantabio, PN95146) according to the manufacturer’s steps. The qPCR reactions were performed on Bio-Rad CFX Connect Real-time PCR detection system using PowerUp SYBR Green Master Mix (Applied Biosystems, A25742) with ROX reference dye (Invitrogen, #12223012). The significance of the data was evaluated by Student’s t-Test in Microsoft EXCEL software. The qRT-PCR figures were generated by GraphPad Prism 8. All RNA experiments were conducted in RNase/DNase-free environment to avoid RNA degradation and DNA contamination.

The transcriptomics RNAseq or direct RNA sequencing data were generated by RNAseq in this study or were downloaded from NCBI-SRA datasets. The RNAseq reads were aligned to Homo sapiens UCSC hg19 (human) (RefSeq gene annotation) or Mus musculus UCSC mm10 (RefSeq gene annotation) using STAR aligner. Differential gene expression was performed by rlog Transformation function from DESeq2. Gene regulation significance was assessed with one-way ANOVA followed by Bonferroni’s post hoc test. The significant regulations (adj. P.Val<0.05) were selected for analysis: (1) Microglia subgroup (DAM, Neurodegeneration, Interferon, Proliferation, LPS-Related) markers or modules analysis were performed according to previous publications [8]. (2) Canonical Pathway, Upstream Analysis, Mechanistic Network of Upstream Regulators, Regulator Effect Network, and Causal Network were performed by Ingenuity Pathways Analysis (IPA, QIAGEN) pathway analysis. Putative upstream Cytokines, Transcriptional factors, Receptors, and Z-Score evaluation of activation or inhibitions of Canonical Pathways (Cellular Immune and Cytokine Signaling) were listed for pathway comparison. (3) KEGG and GO functional category enrichment statistics were performed by DAVID v6.8 [9].

Additional details and understanding may be achieved by reference to experimental data results, described in more detail below.

[image]

FIG. 1 is a schematic diagram of the changes of the downstream signaling pathways by blocking or decreasing STING signaling activation.

[image]

[image]

[image]

The FIGURE presents a collection of charts providing selected experimental data results in one embodiment. As illustrated, the Canonical Pathway analysis of Ingenuity Pathways Analysis (IPA, QIAGEN) revealed that Interferon Signaling is the top signaling pathway in the human AD postmortem brain tissue transcriptomics data (2A). The Upstream Analysis of Ingenuity Pathways Analysis (IPA, QIAGEN) on the human AD postmortem brain tissue transcriptomics data revealed that multiple immune signaling pathways, including the IRF3-IRF7, the STING-TFAF3-TBK1, and the IFNa pathways were all activated, which indicated the Interferon-Related Module of the pathological microglia were enriched in AD postmortem brain (2B). The Mechanistic Network of Upstream Regulators analysis of Ingenuity Pathways Analysis (IPA, QIAGEN) on the human AD postmortem brain tissue transcriptomics data revealed that a pro-inflammatory network (cGAS-Type I Interferon-NFkB-Proinflammatory factors) were activated in in AD postmortem brain (2C).

[image]

The FIGURE presents a collection of charts providing selected experimental data results in one embodiment. The KEGG pathway statistics on the human AD postmortem brain tissue microarray data revealed that the STING-IFN I signaling activation were mainly enriched in hippocampus and prefrontal cortex, and the activation intensity was gradually enhanced during the stage 1 to stage 6 of the AD Braak staging (3A). The qRT-PCR gene expression analysis verified that STING-IFN I signaling were significantly activated in the human AD postmortem brain tissue (3B) .

[image]

[image]

The FIGURE presents a collection of charts providing selected experimental data results in one embodiment. As shown, the Upstream Analysis of Ingenuity Pathways Analysis (IPA, QIAGEN) on the microglial single cell transcriptomics data of the Alzheimer’s Disease 5XFAD mouse model revealed that the STING-TFAF3-TBK1, the IRF3-IRF7, and the IFNa pathways were all activated in the Interferon-Related Module. Of note, the STING-TFAF3-TBK1 pathway is the top original signaling of the cellular subgroup, the Interferon-Related Module (4A). The Canonical Pathway analysis of Ingenuity Pathways Analysis (IPA, QIAGEN) revealed that the Interferon-Related Module may induce autoimmune pathway activation and were mainly induced by cytosolic pattern recognition receptors (PRR) activation in Alzheimer’s Disease 5XFAD mouse model (4B).

[image]

The FIGURE presents a collection of charts providing selected experimental data results in one embodiment. As shown, the Mechanistic Network of Upstream Regulators analysis of Ingenuity Pathways Analysis (IPA, QIAGEN) revealed that the Interferon-Related Module markers of the Alzheimer’s Disease 5XFAD mouse model formed a pro-inflammatory network (IRF3/IRF7-Type I Interferon-NFkB-Proinflammatory factors) (5A). The qRT-PCR gene expression analysis verified that STING-IFN I signaling were significantly activated in the 4.5-monthes old AD 5XFAD mouse model brain tissue. Of note, this activation is early than that of pro-inflammatory activation. (5B). The upregulations of the Interferon-Related Module markers were enriched in 4-months old of hippocampus and cortex, which indicated that the markers were suitable for early diagnosis in AD models (5C).

[image]

[image]

The FIGURE presents a collection of charts providing selected experimental data results in one embodiment. As shown, the major 25 AD risk factor’s mutations formed a pro-inflammatory network, in which the Type I Interferon (IFN beta) connected the pro-inflammatory activation systemically. Note that the mutations may were also involved in peripheral immune activation (6A). In cultured human peripheral blood monocytes (HBMs), transcriptomics analysis revealed that decreasing STING signaling activation using C-178 antagonist treatment can reduce the Interferon Signaling pathway (6B).

[image]

[image]

[image]

[image]

[image]

[image]

The FIGURE presents a collection of charts providing selected experimental data results in one embodiment. As shown, in cultured human peripheral blood monocytes (HBMs), decreasing STING signaling activation using C-178 antagonist treatment can reduce the gene expression levels of the Type I IFN-induced ISGs (hIRF7, hIFIT3, hOASL1, hMX1) (7A, 7B, 7C, 7D), but can not influence the gene expression levels of other AD markers (hIGF1, hAPOE) (7E, 7F). These ISGs regulations indicated the STING signaling was activated in the peripheral blood monocytes (HBMs) of AD patients, and STING activation was a diagnosis marker for AD patients. There were 6 individual donors in each treatment. (* P<0.05, ** P<0.01, *** P<0.001)

### REFERENCES

1. A. C. Cuello, Early and Late CNS Inflammation in Alzheimer’s Disease: Two Extremes of a Continuum? Trends in pharmacological sciences 38, 956-966 (2017).

2. I. Benilova, E. Karran, B. De Strooper, The toxic Abeta oligomer and Alzheimer’s disease: an emperor in need of clothes. Nature neuroscience 15, 349-357 (2012).

3. D. L. Burdette, K. M. Monroe, K. Sotelo-Troha, J. S. Iwig, B. Eckert, M. Hyodo, Y. Hayakawa, R. E. Vance, STING is a direct innate immune sensor of cyclic di-GMP. Nature 478, 515-518 (2011).

4. J. M. Taylor, Z. Moore, M. R. Minter, P. J. Crack, Type-I interferon pathway in neuroinflammation and neurodegeneration: focus on Alzheimer’s disease. Journal of Neural Transmission 125, 797-807 (2018).

5. S. D. Mesquita, A. C. Ferreira, F. Gao, G. Coppola, D. H. Geschwind, J. C. Sousa, M. Correia-Neves, N. Sousa, J. A. Palha, F. Marques, The choroid plexus transcriptome reveals changes in type I and II interferon responses in a mouse model of Alzheimer’s disease. Brain, behavior, and immunity 49, 280-292 (2015).

6. S. M. Haag, M. F. Gulen, L. Reymond, A. Gibelin, L. Abrami, A. Decout, M. Heymann, F. G. van der Goot, G. Turcatti, R. Behrendt, A. Ablasser, Targeting STING with covalent small-molecule inhibitors. Nature 559, 269-273 (2018).

7. K. Srinivasan, B. A. Friedman, J. L. Larson, B. E. Lauffer, L. D. Goldstein, L. L. Appling, J. Borneo, C. Poon, T. Ho, F. Cai, P. Steiner, M. P. van der Brug, Z. Modrusan, J. S. Kaminker, D. V. Hansen, Untangling the brain’s neuroinflammatory and neurodegenerative transcriptional responses. Nature communications 7, 11295 (2016).

8. B. A. Friedman, K. Srinivasan, G. Ayalon, W. J. Meilandt, H. Lin, M. A. Huntley, Y. Cao, S. H. Lee, P. C. G. Haddick, H. Ngu, Z. Modrusan, J. L. Larson, J. S. Kaminker, M. P. van der Brug, D. V. Hansen, Diverse brain myeloid expression profiles reveal distinct microglial activation states and aspects of Alzheimer’s Disease not evident in mouse models. Cell reports 22, 832-847 (2018).

9. D. W. Huang, B. T. Sherman, R. A. Lempicki, Systematic and integrative analysis of large gene lists using DAVID bioinformatics resources. Nature Protocols 4, 44-57 (2009).

What is claimed is:

1. Preparation of products for diagnosis of AD by monitoring STING signaling activation.

2. As described in claim 1, AD can be mild, moderate or severe AD.

3. As described in claim 1, STING signaling pathway refers to either of the CGAS-STING-TBK1-IRF3-IFN I cascade signaling pathway or the CGAS-STING-IKK-NFKB-IFN I cascade signaling pathway or both.

4. The methods for monitoring STING signaling activation refers to qRT-PCR, Digital qPCR, Next generation sequencing (NGS), Single cell RNAseq (scRNAseq), In situ hybridization, Northern blot, Electrophoretic mobility shift assay (EMSA), Proteomics, Western blot, Antibodies staining, Immunohistochemistry, Immunocytochemistry, Immunofluorescence (IF), Bioinformatics, Immunoprecipitation (IP), Co-immunoprecipitation (Co-IP), Enzyme-linked immunosorbent assay (ELISA), Fluorescence resonance energy transfer (FRET), Reporting gene assay, siRNA, miRNA, antisense oligonucleotides, CRISPR/Cas9 gene editing products, antagonists, blockers, small molecule compounds that inhibit the function and/or activity of main molecules of STING signaling pathways, and one of or a combination of promoter elements or expression vectors, which can downregulate the gene, the mRNA, or the protein expression levels of the major molecules of the STING signaling pathway.

5. (canceled)

6. The products include reagents, enzymes, kits, devices, equipment, and their compositions.

7. (canceled)
